(19) United States
(12) Patent Application Publication
(10) Pub. No.: US 2016/0361381 A1
(43) Pub. Date: Dec. 15, 2016

(54) TOPICAL COMPOSITION COMPRISING AN ANTIBACTERIAL AGENT

(71) Applicant: BUZZZ PHARMACEUTICALS LIMITED, Raheny, Dublin (IE)

(72) Inventors: Darren M. GREEN, Cardiff (GB); Kenneth WALTERS, Cardiff (GB)

(21) Appl. No.: 15/120,910

(22) PCT Filed: Feb. 12, 2015
(86) PCT No.: PCT/EP2015/052998

§ 371 Date: Aug. 23, 2016

(30) Foreign Application Priority Data
Feb. 12, 2014 (GB) 1402448.3

(51) Int. Cl. A61K 38/12; A61K 47/34; A61K 47/14; A61K 47/32; A61K 9/16; A61K 9/00

(52) U.S. Cl. CPC A61K 38/12; A61K 9/16; A61K 9/0014; A61K 47/14; A61K 47/32; A61K 47/34

(57) ABSTRACT

The present invention relates to a pharmaceutical composition suitable for topical application to the body and which comprises an antibacterial agent that is dissolved and/or suspended within an oily vehicle. The compositions of the present invention are spontaneously spreadable upon application to a surface of the body. The compositions of the present invention are suitable for the topical treatment of bacterial infections on the skin and/or mucosal surfaces, such as, for example, the topical treatment of Methicillin-resistant Staphylococcus aureus (MRSA), and other complicated skin and skin structure infections (cSSSIs), such as, for example, gangrene.

### FIELD OF THE INVENTION

The present invention relates to a novel formulation. More specifically, the present invention relates to novel antibiotic formulations that are suitable for topical administration. The formulations of the present invention are suitable for the topical treatment of bacterial infections on the skin and/or mucosal surfaces, such as, for example, the topical treatment of Methicillin-resistant Staphylococcus aureus (MRSA), and other complicated skin and skin structure infections (cSSSIs), such as, for example, gangrene.

The present invention therefore also relates to the use of these formulations for the treatment of topical infections (for example MRSA), as well as to processes for the preparation of the formulations defined herein.

### BACKGROUND OF THE INVENTION

Methicillin-resistant Staphylococcus aureus (MRSA) is a bacterium responsible for several problematic and difficult-to-treat infections in humans. It is also called oxacillin-resistant Staphylococcus aureus (ORSA). MRSA is any strain of Staphylococcus aureus that has developed, through the process of natural selection, resistance to beta-lactam antibiotics, which include the penicillins (methicillin, dicloxacillin, nafcillin, oxacillin, etc.) and the cephalosporins. Strains unable to resist these antibiotics are classified as methicillin-sensitive Staphylococcus aureus, or MSSA. The evolution of such resistance does not cause the organism to be more intrinsically virulent than strains of Staphylococcus aureus that have no antibiotic resistance, but resistance does make MRSA infection more difficult to treat with standard types of antibiotics, and therefore more dangerous.

MRSA is especially troublesome in hospitals and nursing homes, where patients with open wounds, invasive devices, and weakened immune systems are at greater risk of infection than the general public.

Staphylococcus aureus is a bacteria found on the skin and in the nostrils of approximately 30% of healthy people. A subset of this population carry the MRSA strain of Staphylococcus aureus on their skin or in their nostrils and are unaware, because it does them no harm and they have no symptoms. This is known as ‘colonisation’. MRSA colonisation tends to occur in the nose, under the arms (axilla), the groin, or perineum (skin between the rectum and genitals).

MRSA can cause particular harm if it is able to enter the body. For example, MRSA can cause serious wound infections, chest infections or infections of the blood stream. As such, MRSA infection is a significant risk factor in major surgery.

Many hospitals now screen patients for MRSA prior to admittance for elective surgical procedures. If a patient is identified as a carrier of MRSA on either their skin or in their nose then a treatment plan can be implemented to eradicate the MRSA prior to the surgery.

As the areas of MRSA colonisation on the skin are typically the groin, the perineal area and the axilla regions, these regions are notoriously difficult to treat using traditional creams and ointments. In particular, it can be hard to get good surface coverage of these areas of the body with a traditional “manually applied” antibiotic formulation.

There is also a need for improved antibiotic formulations to treat other topical infections, such as, for example, impetigo and complicated skin and skin structure infections (cSSSIs). An example of a cSSSI is an infection such as gangrene.

There is therefore a need for improved antibiotic formulations for topical application to eradicate MRSA and other pathogenic bacteria.

In particular, there is a need for a formulation that can be applied to the desired area of the body.

There is also a need for a formulation that can provide better coverage (and therefore treatment) of the infected area, including poorly accessible areas such as the groin, the perineum and axilla regions of the body.

One antibiotic compound that can be used for the treatment of MSRA infection is daptomycin. Daptomycin is a lipopeptide antibiotic that has the structural formula I shown below:

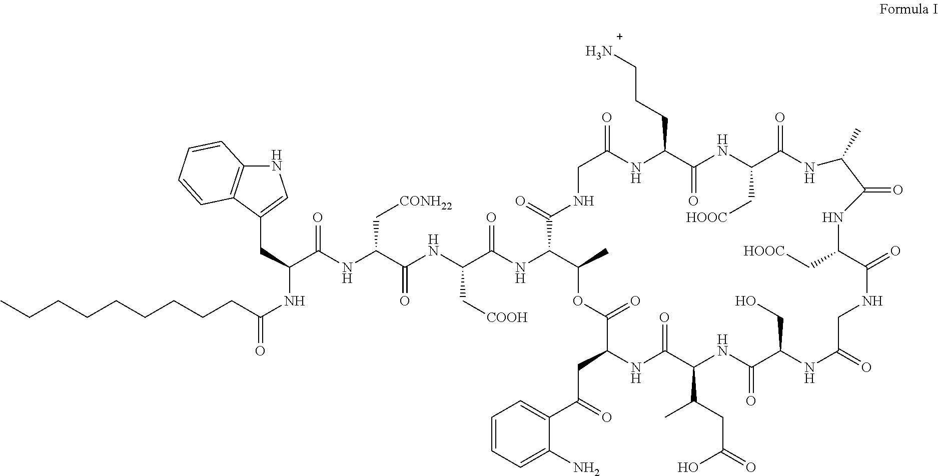

Daptomycin is described in U.S. Pat. No. 4,537,717, the entire contents of which are incorporated herein by reference.

In addition to its use for the treatment of MRSA, daptomycin is also used for the treatment of methicillin-susceptible Staphylococcus aureus (MSSA); streptococcal infections such as Streptococcus pyrogenes, Streptococcus agalactiae, Streptococcus dysgalactiae; and Enterococcus faecalis. Daptomycin is commercially available as an intravenous infusion (Cubicin™), but no topical formulations of daptomycin are available.

Daptomycin is stable in a lyophilised form, but it is susceptible to hydrolysis in aqueous environments.

There is therefore a particular need for improved formulations of daptomycin that are: (i) suitable for topical administration; and (ii) stable.

### SUMMARY OF THE INVENTION

The present invention provides a novel pharmaceutical composition that is suitable for topical application for the treatment of skin infections, such as, for example, MRSA.

Thus, according to a first aspect of the invention, there is provided a pharmaceutical composition suitable for topical application to the body, said composition comprising an antibacterial agent that is dissolved and/or suspended in an oily vehicle as defined herein.

In another aspect of the invention, there is provided a topical pharmaceutical composition comprising an antibacterial agent that is dissolved and/or suspended in an oily vehicle as defined herein.

As defined further herein, the oily vehicle is suitably a low viscosity fluid that is capable of spontaneously spreading on the skin or mucosal surfaces to which is it is applied. This means that it is not necessary to manually spread the composition on the skin or mucosal surface to which it is applied, although manual spreading may additionally be used if desired.

Thus, in another aspect, the present invention provides a pharmaceutical composition suitable for topical application to the body, said composition comprising an antibacterial agent that is dissolved and/or suspended within an oily vehicle;

wherein the pharmaceutical composition is spontaneously spreadable upon application to a surface of the body.

In another aspect, the present invention provides a topical pharmaceutical composition comprising an antibacterial agent that is dissolved and/or suspended within an oily vehicle;

wherein the pharmaceutical composition is spontaneously spreadable upon application to a surface of the body.

In another aspect, the present invention provides a pharmaceutical composition suitable for topical application to the body, said composition comprising:
(i) an antibacterial agent;
(ii) an oily vehicle; and
(iii) optionally a suspending agent and/or a surfactant, wherein the suspending agent and/or surfactant renders the antibacterial agent dispersible in the oily vehicle;

and wherein the pharmaceutical composition is spontaneously spreadable upon application to the a surface of the body.

In another aspect, the present invention provides a topical pharmaceutical composition, said composition comprising:
(i) an antibacterial agent;
(ii) an oily vehicle; and
(iii) optionally a suspending agent and/or a surfactant, wherein the suspending agent and/or surfactant renders the antibacterial agent dispersible in the oily vehicle;
and wherein the pharmaceutical composition is spontaneously spreadable upon application to the a surface of the body.

In another aspect, the present invention provides a pharmaceutical composition as defined herein for use in the treatment of topical infections (e.g. infections present on the skin or accessible mucosal surfaces, such as the nasal passages).

In another aspect, the present invention provides a method of treating a topical infection, said method comprising administering to a human or animal subject in need of such treatment a therapeutically effective amount of a pharmaceutical composition as defined herein.

In a particular aspect, the present invention provides a pharmaceutical composition as defined herein for use in the treatment of MRSA, impetigo or other complicated skin and skin structure infections (cSSSIs).

In another particular aspect, the present invention provides a method of treating MRSA, impetigo or other complicated skin and skin structure infections (cSSSIs), said method comprising administering to a human or animal subject in need of such treatment a therapeutically effective amount of a pharmaceutical composition as defined herein.

In another aspect, the present invention provides a pharmaceutical composition as defined herein for use in the eradication of MRSA prior to surgery.

In another aspect, the present invention provides a method of eradicating MRSA prior to surgery, said method comprising administering to a human or animal subject in need of such treatment a therapeutically effective amount of a pharmaceutical composition as defined herein.

In another aspect, the present invention provides a method of preparing a pharmaceutical composition as defined herein, said method comprising suspending an antibacterial agent as defined herein in an oily vehicle.

In another aspect, the present invention provides a device comprising a pharmaceutical composition as defined hereinbefore, wherein the device is configured to dispense a volume of the composition (for example, in the form of a spray, jet, or one or more droplets or fixed bolus, optionally for application via some form of applicator utensil).

### DETAILED DESCRIPTION OF THE INVENTION

### Definitions

Unless otherwise stated, the following terms used in the specification and claims have the following meanings set out below.

The terms “treating” or “treatment” include prophylaxis as well as the alleviation of established symptoms of a disease or condition. “Treating” or “treatment” therefore includes: (1) preventing or delaying the appearance of clinical symptoms of the disease or condition developing in a subject that may be afflicted with or predisposed to the disease or condition, but does not yet experience or display clinical or subclinical symptoms of the disease or condition, (2) inhibiting the disease or condition, i.e., arresting, reducing or delaying the development of the disease or condition or a relapse thereof (in case of maintenance treatment) or at least one clinical or subclinical symptom thereof, or (3) relieving or attenuating the disease or condition, i.e., causing regression of the disease or condition or at least one of its clinical or subclinical symptoms.

The term “topical pharmaceutical composition” is used herein to mean a pharmaceutical composition adapted and suitable for topical application.

The term “topical application” means application to the body surfaces, including the skin, mucous membranes (e.g. the nasal passages) and any sites of injury or wounds present on the in the skin or mucous membranes.

The term “spontaneously spreadable” is used herein to define the pharmaceutical compositions of the present invention and means that, upon topical application (as defined above), the composition spreads spontaneously from the point of application. The oily vehicle is suitably a low viscosity fluid that spreads over the skin or mucosal surface following application. This means that it is not necessary to manually spread the composition on the skin or mucosal surface to which it is applied, although manual spreading may additionally be used if desired. Suitably, on contact with the skin/mucosa, the composition will spread across the surface of the skin/mucosa to cover an area that is at least three-fold larger, and more suitably five-fold larger, than the area covered by a droplet of water of equivalent volume. Suitably the spreading to cover an area that is at least three-fold larger, and more suitably five-fold larger, than the area covered by a droplet of water of equivalent volume occurs within a short time period of time, for example, up to 5 minutes, or more preferably up to 2 minutes.

The term “D90” is used herein to refer to the particle size distribution of antimicrobial agent particles. D90 is the value of the particle diameter at 90% in the cumulative distribion of particles sizes present. For example, if D90=50 μm, then 90% of the particles in the sample are smaller than 50 μm and 10% are larger than 50 μm.

### Pharmaceutical Compositions

As previously stated, the present invention provides novel pharmaceutical compositions that are suitable for topical application to the body to treat infections of the skin and mucosal surfaces, such as, for example, MRSA.

In particular, the present invention provides a pharmaceutical composition suitable for topical application to the body, said composition comprising an antibacterial agent that is dissolved and/or suspended in an oily vehicle as defined herein.

In another aspect of the invention, there is provided a topical pharmaceutical composition comprising an antibacterial agent that is dissolved and/or suspended in an oily vehicle as defined herein.

In another aspect, the present invention provides a pharmaceutical composition suitable for topical application to the body, said composition comprising an antibacterial agent that is dissolved and/or suspended within an oily vehicle;

wherein the pharmaceutical composition is spontaneously spreadable upon application to a surface of the body.

In another aspect, the present invention provides a topical pharmaceutical composition comprising an antibacterial agent that is dissolved and/or suspended within an oily vehicle;

wherein the pharmaceutical composition is spontaneously spreadable upon application to a surface of the body.

Suitably, the antibacterial agent is suspended within the oily vehicle. Suitably, the antimicrobial agent is uniformly dispersed or dispersible within the oily vehicle. In this context, the terms “dispersed or dispersible” mean that the antimicrobial agent is either maintained in the form of a homogenous dispersion within the oily vehicle or that a homogenous dispersion can be easily attained by agitating the composition (e.g. shaking the composition prior to use).

Suitably, the composition is a liquid at room temperature and at skin temperature (34-38° C.).

The pharmaceutical compositions of the present invention possess a number of advantageous properties.

Firstly, the use of an oily vehicle renders the compositions of the invention ideally suited to antimicrobial agents that are susceptible to hydrolysis in aqueous environments (such as, for example, daptomycin). Thus, commercially viable topical formulations with good stability can be prepared.

As indicated above, the antimicrobial agent is either uniformly dispersed in the oily vehicle or dispersible in the vehicle, so a homogenous suspension can be easily attained by agitating (e.g. shaking) the composition prior to use.

The spreadable nature of the pharmaceutical composition, which is primarily imparted by the nature of the oily vehicle, also enables the compositions to spontaneously spread across the desired portion of the body following application. This property of the compositions means that there is less need for a patient or carer to manually spread the formulation over the affected area of the body. This provides a number of advantages:
There is a lower risk of the infection (e.g. MRSA) spreading if manual contact can be avoided or at least minimised.
Controlled doses of the composition can be applied, for example as a spray or spot on application of a defined volume.
The uniformity (and low viscosity) with which the composition covers the affected area of the body can be improved (manual spreading can inherently provide poor uniformity of coverage).
Compared to traditional topical preparations, the compositions of the present invention can improve the treatment of infections that reside in poorly accessible part of the body, such as, for example, the groin, the perineal area and the axilla regions (which are a particular problem in the case of MRSA).

The formulations of the present invention also exhibit good emolliency.

### Antimicrobial Agent

The antimicrobial agent present in the pharmaceutical compositions of the present invention may be any antimicrobial agent suitable for topical use.

The compositions of the present invention are particularly suited to antimicrobial agents that have poor stability in aqueous media (but the scope of the present invention should not be construed as being limited to such antimicrobial agents).

The antimicrobial agents used in the compositions of the present invention will typically have little or no solubility in the oily vehicle of the composition. As such, the antimicrobial agent is present as a dispersion or suspension within the oily vehicle.

The antimicrobial agent may be present as a pharmaceutically acceptable salt or solvate. Accordingly, references herein to antimicrobial agents (either as generically or by reference to specifically named antimicrobial agent) will include, where appropriate, any pharmaceutically acceptable salts or solvates thereof.

In an embodiment of the invention, the antimicrobial agent is present as discrete particles.

Suitably, the antimicrobial agent is in a fine particulate form, suitably a microparticulate or nanoparticulate form.

Suitably, the particles of antimicrobial agent have a D90 of 75 μm or less, more suitably a D90 of 60 μm or less, more suitably D90 of 50 μm or less, and even more suitably less than 40 μm or less.

The antimicrobial agent may be present at any suitable amount in the composition of the invention. Suitably, the antimicrobial agent is present in an amount of 0.01 wt. % to 10 wt. %, more suitably 0.05 wt. % to 5 wt. %, even more suitably 0.1 wt. % to 4 wt. %, even more suitably 0.2 wt. % to 4 wt. %, and most suitably 0.5 wt. % to 3 wt. %.

A person skilled in the art will be able to select appropriate antimicrobial agents for inclusion in the compositions of the present invention by virtue of their physicochemical properties and their potential use for the treatment of topical infections. Some examples of suitable antimicrobial agents include daptomycin, vancomycin, mupirocin, polymixin B, clindamycin, retapamulin, neomycin, bacitracin and fusidic acid, or pharmaceutically acceptable salts thereof.

In an embodiment of the invention, the pharmaceutical composition may comprise two or more antimicrobial agents, for example agents selected from those mentioned herein.

In an embodiment of the invention, an antimicrobial agent is selected from the group consisting of daptomycin, vancomycin, mupirocin, polymixin B, clindamycin, retapamulin, neomycin, bacitracin and fusidic acid or pharmaceutically acceptable salts thereof.

In a further embodiment of the invention, an antimicrobial agent is selected from daptomycin, vancomycin and/or mupirocin.

In a particular embodiment of the invention, the antimicrobial agent is daptomycin, or a pharmaceutically acceptable salt thereof. For the avoidance of doubt, and as previously stated above, references herein to daptomycin should be construed as covering daptomycin or any pharmaceutically acceptable salt or solvate thereof. Suitably, the average particle size of the daptomycin is less than 50 μm, more suitably less than 40 μm, and even more suitably less than 25 μm. Suitably, the daptomycin is present in an amount of 0.01 wt. % to 10 wt. % of the composition. more suitably 0.05 wt. % to 5 wt. %, even more suitably 0.1 wt. % to 4 wt. %, and most suitably 0.5 wt. % to 3 wt. %.

In a particular embodiment, the antimicrobial agent is daptomycin present at a concentration of 2+/−0.5 wt. % of the composition.

### Oily Vehicle

The oily vehicle provides the medium in which the antimicrobial agent or agents is/are dissolved/suspended/dispersed. The oily vehicle also imparts the spreadability characteristics of the compositions of the present invention.

In embodiments where the antimicrobial agent is suspended or dispersed in the oily vehicle, any suitable oily vehicle in which the antimicrobial agent is insoluble or poorly soluble, and that is “spontaneously spreadable” as defined herein, may be used in the compositions of the present invention.

One or more oil components may be present in the oily vehicle. In an embodiment, the oily vehicle comprises one or more oil components selected from the group consisting of:
mono-, di- and/or triglycerides comprising glycerol and (6-18C) fatty acids;
propylene glycol esters of (6-18C) fatty acids;
(6-18C) fatty acid esters (e.g. isopropyl myristate);
(6-18C) fatty alcohols;
silicone fluids/oils (such as, for example dimethicone, hexamethyldisiloxane, octamethyltrisiloxane and cyclomethicone); and
lanolin/lanolin derivatives.

Particular examples of (6-18C) fatty acids include caprylic acid, capric acid, lauric acid, myristic acid, sebacic, and oleic acid.

In an embodiment, the fatty acid components present in the oily vehicle are (8-18C) fatty acids.

Particular examples of glycerides that may be used in the compositions of the present invention include glycerol monooleate (e.g. Capmul® GMO-50), glycerol monocaprylate/caprate (e.g. Capmul® MCM), and caprylic/capric triglyceride (e.g. Captex® 355).

Particular examples of propylene glycol esters of (6-18C) fatty acids include propylene glycol monocaprylate (Capyrol® 90) and propylene glycol dicaprylocaprate (Labrafac® PG).

Particular examples of (6-18C) fatty acid esters, include (8-18C) fatty acid (1-6C) alkyl esters, such as, for example, isopropyl myristate, dioctyladipate, isobutyl palmitate, octyldodecyl cocoate, and/or diisopropyl sebacate.

Particular examples of (6-18C) fatty alcohols include capric alcohol, lauryl alcohol, myristyl alcohol, cetyl alcohol, palmitoleyl alcohol and steryl alcohol.

Particular examples of silicone fluids/oils include dimethicone, hexamethyldisiloxane, octamethyltrisiloxane and cyclomethicone.

Particular examples of lanolin/lanolin derivatives that may be used in the compositions of the present invention include acetylated lanolin (e.g. Modulan®).

The oily vehicle typically forms the largest proportion of the compositions of the present invention. Typically, the oily vehicle will constitute between 70 wt. % and 99.5 wt. % of the composition. More typically, it will constitute 80-95 wt. % of the composition and even more typically between 85 and 93 wt. % of the composition.

In certain embodiments of the invention, the oily vehicle comprises one or more oil components selected from the group consisting of:
mono-, di- and/or triglycerides comprising glycerol and (6-18C) fatty acids;
propylene glycol esters of (6-18C) fatty acids;
(6-18C) fatty acid esters (e.g. isopropyl myristate); and
(6-18C) fatty alcohols.

In another embodiment, the oily vehicle comprises one or more oil components selected from the group consisting of:
mono-, di- and/or triglycerides comprising glycerol and (6-18C) fatty acids;
propylene glycol esters of (6-18C) fatty acids; and
(6-18C) fatty acid esters (e.g. isopropyl myristate).

In some further embodiments, it advantageous to add further oily emollients to the oily vehicle. The additional emollients may constitute up to 70% wt. % of the composition and more suitably up to 65 wt. % of the composition. Any suitable oily emollients may be used in the compositions of the present invention. Examples of suitable emollient components include silicone fluids/oils (such as, for example dimethicone, hexamethyldisiloxane, octamethyltrisiloxane and cyclomethicone); lanolin/lanolin derivatives; cocoyl adipic acid/trimethylproprane copolymer, adipate fatty acid esters such as diisobutyl adipate, (6C fatty acid esters) fatty acid esters of citric acid, e.g. tributylcitrate, and volatile oils, e.g. peppermint oil.

In a particular embodiment, the oily vehicle comprises one or more oil components selected from the group consisting of:
mono-, di- and/or triglycerides comprising glycerol and (6-18C) fatty acids;
propylene glycol esters of (6-18C) fatty acids;
(6-18C) fatty acid esters (e.g. isopropyl myristate); and
silicone fluids/oils (such as, for example dimethicone, hexamethyldisiloxane and cyclomethicone).

In a further embodiment, the oily vehicle comprises (the ranges refer to the wt. % of the total pharmaceutical composition of the invention):
5-45 wt. % (6-18C) fatty acid esters (e.g. isopropyl myristate);
5-60 wt. % mono-, di- and/or triglycerides comprising glycerol and (6-18C) fatty acids;
0-10 wt. % propylene glycol esters of (6-18C) fatty acids;
0-70 wt. % silicone fluids/oils (such as, for example dimethicone, hexamethyldisiloxane, octamethyltrisiloxane and cyclomethicone); and
0-5 wt. % lanolin/lanolin derivative.

In a further embodiment, the oily vehicle comprises (the ranges refer to the wt. % of the total pharmaceutical composition of the invention):
10-20 wt. % (6-18C) fatty acid esters (e.g. isopropyl myristate);
10-30 wt. % mono-, di- and/or triglycerides comprising glycerol and (8-18C) fatty acids;
0-10 wt. % propylene glycol esters of (6-18C) fatty acids; and
0-70 wt. % silicone fluids/oils (such as, for example dimethicone, hexamethyldisiloxane, octamethyltrisiloxane and cyclomethicone).

In a further embodiment, the oily vehicle comprises (the ranges refer to the wt. % of the total pharmaceutical composition of the invention):
10-20 wt. % (6-18C) fatty acid esters (e.g. isopropyl myristate);
10-30 wt. % mono-, di- and/or triglycerides comprising glycerol and (6-18C) fatty acids;
0-10 wt. % propylene glycol esters of (6-18C) fatty acids; and
40-70 wt. % silicone fluids/oils (such as, for example dimethicone, hexamethyldisiloxane, octamethyltrisiloxane and cyclomethicone).

In a further embodiment, the oily vehicle comprises (the ranges refer to the wt. % of the total pharmaceutical composition of the invention):
10-20 wt. % (6-18C) fatty acid esters (e.g. isopropyl myristate);
10-20 wt. % mono-, di- and/or triglycerides comprising glycerol and (6-18C) fatty acids; and
40-70 wt. % silicone fluids/oils (such as, for example dimethicone, hexamethyldisiloxane, octamethyltrisiloxane and cyclomethicone).

In a further embodiment, the oily vehicle comprises (the ranges refer to the wt. % of the total pharmaceutical composition of the invention):
10-40 wt. % isopropyl myristate;
10-60 wt. % caprylic/capric triglyceride/medium chain triglycerides;
0-30 wt. % dimethicone;
0-40 wt. % hexamethyldisiloxane; and
0-30 wt. % cyclomethicone.

In a further embodiment, the oily vehicle comprises (the ranges refer to the wt. % of the total pharmaceutical composition of the invention):
10-20 wt. % isopropyl myristate;
10-20 wt. % caprylic/capric triglyceride/medium chain triglycerides;
10-20 wt. % dimethicone;
10-40 wt. % hexamethyldisiloxane; and
10-30 wt. % cyclomethicone.

### Suspending Agent

In certain embodiments of the invention, the pharmaceutical composition further comprises a suspending agent. A suspending agent functions to help prevent the aggregation of the antibacterial agent particles suspended in the oily vehicle. Therefore, a suspending agent may be used to enable the antimicrobial particles to either remain homogenously dispersed within the oily vehicle over time or enable a homogenous dispersion to be easily attained by agitating (e.g. shaking) the composition.

The suspending agent suitably has little or no solubility in the oily vehicle.

Particularly suitable suspending agents include one or more of the following:
cross-linked polymers suitably in a micronized form, e.g. PVP (crospovidone) & sodium croscarmellose;
clays: micronized clays aluminium phyllosilicate (bentonite);
magnesium/aluminium silicates;
silicas: silica (e.g. Syliod®, Cab-o-sil®), silicon dioxide (e.g. Aerosil®); and
hydrogenated oils: trihydroxystearin (Thixcin® R)

In a particular embodiment, the suspending agent is micronized crospovidone (e.g. Kollidon® CL-M). This material also functions as a water scavenger, and thus may improve the stability of any water-sensitive components.

The suspending agent is present in an amount sufficient to suspend the antibacterial agent. Typically, the suspending agent will be present in an amount of 0.2-15 wt. %, more typically 0.2-10 wt. % and most typically at 0.2-7 wt. %.

### Surfactants

The oily vehicle may further comprise one or more oily surfactants. The amount of such oily surfactants present will typically be up to 7.5 wt. % of the total composition, more typically up to 5 wt. % and most typically up to 3 wt. %.

Such surfactants suitably have a HLB (hydrophilic-lipophilic balance) value of less than 7.

Examples of suitable surfactants include polyoxyethylated fatty acid (10-18C) esters of sorbitan (e.g. Span 20, Span 80); (8-18C) fatty acid esters, and polyoxypropylene/polyoxyethylene block co-polymers (Poloxamers®).

In certain embodiments of the invention, the pharmaceutical composition comprises a suspending agent as defined above and an oily surfactant as defined herein.

### Stabiliser

In certain embodiments of the invention, the composition may further comprise one or more stabilisers.

In embodiments where the pharmaceutical composition comprises daptomycin, a divalent metal salt, such as calcium and/or magnesium salts, may be added. Examples of suitable salts to add to the compositions are calcium and/or magnesium fatty acid salts (e.g. calcium and/or magnesium stearate), calcium and/or magnesium sulphate, calcium and/or magnesium trisilicate, and calcium and/or magnesium carbonate/hydroxide In a particular embodiment, calcium stearate is used as the stabiliser.

The amount of stabiliser present may be 0-5 wt. % of the total composition, for example 0.5 to 3 wt. %.

### Moisture Content

Suitably the moisture content of the compositions of the present invention is less than 5 wt. %, preferably less than 2 wt. %, more preferably less than 1 wt. %, and most preferably less than 0.5 wt. %.

The moisture content will typically arise as a consequence of the small amounts of moisture that may be present in certain excipients used to prepare the compositions of the invention.

### PARTICULAR EMBODIMENTS OF THE INVENTION

In an embodiment of the present invention, the pharmaceutical composition comprises:
(i) 0.05-5 wt. % of an antibacterial agent;
(ii) 75-99.5 wt. % of an oily vehicle; and
(iii) optionally 0.5-10 wt. % of a suspending agent.

In a further embodiment of the present invention, the pharmaceutical composition comprises:
(i) 1-3 wt. % of an antibacterial agent;
(ii) 80-98 wt. % of an oily vehicle; and
(iii) optionally 1-8 wt. % of a suspending agent.

In another embodiment of the present invention, the pharmaceutical composition comprises:
(i) 1-3 wt. % of an antibacterial agent;
(ii) 80-96 wt. % of an oily vehicle; and
(iii) optionally 3-7 wt. % of a suspending agent.

In another embodiment of the present invention, the pharmaceutical composition comprises:
0.05-5 wt. % of daptomycin or a pharmaceutically acceptable salt thereof;
5-45 wt. % (6-18C) fatty acid esters (e.g. isopropyl myristate);
5-60 wt. % mono-, di- and/or triglycerides comprising glycerol and (6-18C) fatty acids;
0-10 wt. % propylene glycol esters of (6-18C) fatty acids;
0-70 wt. % silicone oils (such as, for example dimethicone, hexamethyldisiloxane, octamethyltrisiloxane and cyclomethicone);
0-5 wt. % lanolin/lanolin derivative;
up to 7.5 wt. % of a surfactant selected from polyoxyethylated fatty acid (10-18C) esters of sorbitan (e.g. Span 20, Span 80); (8-18C) fatty acid esters, and polyoxypropylene/polyoxyethylene block co-polymers (Poloxamers®);
0.5-10 wt. % of a suspending agent selected from one or more of the following:
   cross-linked polymers suitably in a micronized for, e.g. PVP (crospovidone) & sodium croscarmellose;
   clays: micronized clays aluminium phyllosilicate (bentonite); magnesium/aluminium silicates;
   silicas: silica (Syliod®, Cab-o-sil®), silicon dioxide (Aerosil®); and
   hydrogenated oils: trihydroxystearin (Thixcin® R); and
0-5 wt. % of a divalent metal salt.

In another embodiment of the present invention, the pharmaceutical composition comprises:
1-5 wt. % of daptomycin or a pharmaceutically acceptable salt thereof;
5-45 wt. % (6-18C) fatty acid esters (e.g. isopropyl myristate);
5-60 wt. % mono-, di- and/or triglycerides comprising glycerol and (6-18C) fatty acids;
0-10 wt. % propylene glycol esters of (6-18C) fatty acids;
0-70 wt. % silicone oils (such as, for example dimethicone, hexamethyldisiloxane, octamethyltrisiloxane and cyclomethicone);
0-5 wt. % lanolin/lanolin derivative;
0-5 wt. % of a surfactant selected from polyoxyethylated fatty acid (10-18C) esters of sorbitan (e.g. Span 20, Span 80); (8-18C) fatty acid esters, and polyoxypropylene/polyoxyethylene block co-polymers (Poloxamers®);
1-8 wt. % of a suspending agent selected from one or more of the following:
   cross-linked polymers suitably in a micronized for, e.g. PVP (crospovidone) & sodium croscarmellose;
   clays: micronized clays aluminium phyllosilicate (bentonite);
   magnesium/aluminium silicates;
   silicas: silica (Syliod®, Cab-o-sil®), silicon dioxide (Aerosil®); and
   hydrogenated oils: trihydroxystearin (Thixcin® R); and
1-3 wt. % of a divalent metal salt.

In another embodiment of the present invention, the pharmaceutical composition comprises:
1-5 wt. % of daptomycin or a pharmaceutically acceptable salt thereof;
5-45 wt. % (6-18C) fatty acid esters (e.g. isopropyl myristate);
5-60 wt. % mono-, di- and/or triglycerides comprising glycerol and (8-18C) fatty acids;
0-70 wt. % silicone oils (such as, for example dimethicone, hexamethyldisiloxane, octamethyltrisiloxane and cyclomethicone);
1-5 wt. % of a surfactant selected from polyoxyethylated fatty acid (10-18C) esters of sorbitan (e.g. Span 20, Span 80); (8-18C) fatty acid esters, and polyoxypropylene/polyoxyethylene block co-polymers (Poloxamers®);
1-8 wt. % of a suspending agent selected from one or more of the following:
   cross-linked polymers suitably in a micronized for, e.g. PVP (crospovidone) & sodium croscarmellose;
   clays: micronized clays aluminium phyllosilicate (bentonite);
   magnesium/aluminium silicates;
   silicas: silica (Syliod®, Cab-o-sil®), silicon dioxide (Aerosil®); and
   hydrogenated oils: trihydroxystearin (Thixcin® R); and
1-3 wt. % of a divalent calcium and/or magnesium salt.

In another embodiment of the present invention, the pharmaceutical composition comprises:
1-5 wt. % of daptomycin or a pharmaceutically acceptable salt thereof;
10-20 wt. % (6-18C) fatty acid esters (e.g. isopropyl myristate);
10-20 wt. % mono-, di- and/or triglycerides comprising glycerol and (6-18C) fatty acids;
50-70 wt. % silicone oils (such as, for example dimethicone, hexamethyldisiloxane, octamethyltrisiloxane and cyclomethicone);
1-5 wt. % of a surfactant selected from polyoxyethylated fatty acid (10-18C) esters of sorbitan (e.g. Span 20, Span 80); (8-18C) fatty acid esters, and polyoxypropylene/polyoxyethylene block co-polymers (Poloxamers®);
1-8 wt. % of crospovidone, sodium croscarmellose and/or trihydroxystearin; and
1-3 wt. % of a divalent calcium and/or magnesium salt.

In another embodiment of the present invention, the pharmaceutical composition comprises:
1-3 wt. % of daptomycin or a pharmaceutically acceptable salt thereof;
10-20 wt. % (6-18C) fatty acid esters (e.g. isopropyl myristate);
10-20 wt. % mono-, di- and/or triglycerides comprising glycerol and (6-18C) fatty acids;
50-70 wt. % silicone oils (such as, for example dimethicone, hexamethyldisiloxane, octamethyltrisiloxane and cyclomethicone);
1-5 wt. % of a surfactant selected from polyoxyethylated fatty acid (10-16C) esters of sorbitan (e.g. Span 20, Span 80); (8-16C) fatty acid esters, and polyoxypropylene/polyoxyethylene block co-polymers (Poloxamers®);
1-8 wt. % of crospovidone, sodium croscarmellose and/or trihydroxystearin; and
1-3 wt. % of a divalent calcium and/or magnesium salt.

In another embodiment of the present invention, the pharmaceutical composition comprises:
1-3 wt. % of daptomycin or a pharmaceutically acceptable salt thereof;
10-40 wt. % isopropyl myristate;
10-60 wt. % caprylic/capric triglyceride/medium chain triglyceride;
0-30 wt. % dimethicone;
0-40 wt. % hexamethyldisiloxane;
0-30 wt. % cyclomethicone;
1-5 wt. % of a surfactant selected from polyoxyethylated fatty acid (10-16C) esters of sorbitan (e.g. Span 20, Span 80); (8-16C) fatty acid esters, and polyoxypropylene/polyoxyethylene block co-polymers (Poloxamers®);
1-8 wt. % of crospovidone, sodium croscarmellose and/or trihydroxystearin; and
1-3 wt. % of a divalent calcium and/or magnesium salt.

In another embodiment of the present invention, the pharmaceutical composition comprises:
1-3 wt. % of daptomycin or a pharmaceutically acceptable salt thereof;
10-20 wt. % isopropyl myristate;
10-20 wt. % caprylic/capric triglyceride/medium chain triglyceride;
10-20 wt. % dimethicone;
10-40 wt. % hexamethyldisiloxane;
10-30 wt. % cyclomethicone;
1-5 wt. % of a surfactant selected from polyoxyethylated fatty acid (10-16C) esters of sorbitan (e.g. Span 20, Span 80); (8-16C) fatty acid esters, and polyoxypropylene/polyoxyethylene block co-polymers (Poloxamers®);
1-8 wt. % of crospovidone, sodium croscarmellose and/or trihydroxystearin; and
1-3 wt. % of a divalent calcium and/or magnesium salt.

### Preparation of the Pharmaceutical Compositions

The pharmaceutical compositions of the present invention can be prepared using conventional techniques known in the art.

The pharmaceutical compositions are suitably prepared by mixing (e.g. homogenising) all of the components together. If any volatile components are present as part of the oily vehicle (e.g. hexamethyldisiloxane or cyclomethicone), then it may be desirable to add these components following the initial mixing of the non-volatile components.

The individual components may be mixed (e.g. homogenised) by simply adding all of the components (optionally excluding any volatile components) at the same time into a mixing vessel and then mixing them all together (a “one-pot” mixture). Alternatively, the components may be added sequentially in two or more steps or stages.

Other experimental conditions requited to prepare the compositions of the present invention, such as homogenisation times, homogenisation speeds, temperature control etc., can be readily determined by a person of ordinary skill in the art.

Further experimental details will also be evident from the accompanying Examples.

Once formed, the pharmaceutical compositions of the present invention may be loaded into a suitable delivery device, which may a metered dosage spray device or a device configured to dispense one or more droplets of the composition when actuated. Suitably, the device is a spray device.

### Delivery Devices

The pharmaceutical compositions of the present invention may be loaded into a suitable delivery device for topical administration.

The selection of the delivery device will depend on the ultimate therapeutic application and the nature of the delivery required. In most cases, it is envisaged that the pharmaceutical compositions of the present invention will be administered as spot on or spray on formulations. As such, devices configured to deliver one or more defined volumes of the composition in the form of droplets, sprays or bolus of the composition of the pharmaceutical compositions are generally preferred.

Thus, in a particular aspect, the present invention provides a device comprising a pharmaceutical composition as defined hereinbefore, wherein the device is configured to dispense a defined volume of the composition (for example, as a spray, jet, one or more droplets or bolus of the composition).

The ability of the pharmaceutical compositions to spontaneously spread across the surface of the skin or mucosal surface to which it is applied means that a suitable spray of the pharmaceutical composition can provide a substantially uniform distribution of the antimicrobial agent on the skin surface.

### Therapeutic Uses

The pharmaceutical compositions of the present invention are particularly suited to the treatment of topical infections. Once administered, the composition will spread across the surface of the skin and/or mucosal surface as previously described and the antimicrobial agent will then be released from the oily vehicle and dissolve in moisture present on the surface of the skin/mucosa.

The diseases that can be treated with the pharmaceutical compositions of the present invention will depend on the antimicrobial agent(s) selected for inclusion in the composition.

Examples of topical infections include impetigo, MRSA and CSSSI's (such as, for example, gangrene).

In embodiments where the antimicrobial agent is, or comprises, daptomycin, the pharmaceutical composition may be used for the treatment of infections caused by Gram-positive bacteria, especially Staphylococcus aureus bacteraemia (e.g. MRSA). In addition to its use for the treatment of MRSA, daptomycin is also used for the treatment of methicillin-susceptible Staphylococcus aureus (MSSA); streptococcal infections such as Streptococcus pyrogenes, Streptococcus agalactiae, Streptococcus dysgalactiae; and Enterococcus faecalis.

Daptomycin-containing compositions of the invention may also be used for the treatment of complicated skin and skin structure infections (cSSSIs) caused by complicated wound infections, such gangrene (“wet” and “dry” gangrene). Dry gangrene begins at the distal part of the limb due to ischemia, and often occurs in the toes and feet of elderly patients due to arteriosclerosis and thus, is also known as senile gangrene. Wet gangrene occurs in naturally moist tissue and organs such as the mouth, bowel, lungs, cervix, and bedsores occurring on body parts such as the sacrum, buttocks, and heels. Wet gangrene is characterised by numerous bacteria and has a poor prognosis (compared to dry gangrene) due to septicemia. In wet gangrene, the tissue is infected by saprogenic microorganisms (Clostridium perfringens or Bacillus fusiformis, for example), which cause tissue to swell and emit a fetid smell.

Daptomycin-containing compositions could also be used alone or in combination with other antibiotics, e.g. polymixin B, to yield a broad spectrum of activity.

A particular application of pharmaceutical compositions of the invention comprising daptomycin is the treatment of MRSA infection. MRSA infection is major issue in hospitals and there is a pressing need for pharmaceutical compositions that can be used topically to treat MRSA present on the skin or mucosal surfaces. There is a particular need for topical pharmaceutical compositions that can be used to eradicate MRSA from the skin or mucosal surfaces prior to elective surgical procedures.

Pharmaceutical compositions of the invention comprising other antibacterial agents may be used for the treatment of any topical infections that are sensitive to antimicrobial agent concerned. For example, pharmaceutical compositions comprising vancomycin may also be used for the treatment of topical infection caused by Gram-positive bacteria. Pharmaceutical compositions comprising mupirocin may also be used for the treatment of topical infection caused by Gram-positive bacteria such as MRSA and MSSA.

The dosage required will vary depending on the antimicrobial agent and its intended use. The actual dosage levels of the antimicrobial agents in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the method of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved. The pharmaceutical compositions of the invention may also be administered on a regimen of 1 to 4 times per day, e.g. once or twice per day. The dosage regimen may be adjusted to provide the optimal therapeutic response.

Suitably, the appropriate dosage can be easily administered by means of dosage device as previously described.

The pharmaceutical compositions of the present invention may be used on their own as the sole therapy. Alternatively, the compositions may be administered as part of a combination therapy with one or more other antimicrobial treatments. Such conjoint treatment may be achieved by way of the simultaneous, sequential or separate administration of the individual components of the treatment.

### EXAMPLES

### Materials

The following materials were used in the preparation of the formulations described herein:

TABLE 1
Materials

| Component | Chemical/common name | Supplier |
|---|---|---|
| Daptomycin | Daptomycin | |
| Labrafac PG | Propylene glycol dicaprylate/ | Gattefosse |
| | dicaprate | |
| IPM | Isopropylmyristate, Isopropyl | Mistral |
| | tetradecanoate | Chemicals |
| Labrafac lipophile | Labrafac Lipophile WL 1349, | Gattefosse |
| | Medium chain triglycerides | |
| Capryol 90 | Propylene glycol monocaprylate | Gattefosse |
| Capmul GMO-50 | Glycerol monooleate | Abitec |
| Captex 355 | Triglycerides of caprylic/capric | Abitec |
| | acid | |
| Ca stearate | Calcium stearate, stearic acid | Sigma-Aldrich |
| | calcium salt | |
| Kollidon CL-M | Crosslinked polyvinylpyrrolidone, | BASF |
| | micronized (Crospovidone) | |
| Span 20 | Sorbitan monolaurate | Croda |
| Span 80 | Sorbitan monooleate | Croda |
| Tween 80 | Polyethylene glycol sorbitan | Croda |
| | monooleate | |
| Thixcin R | 1,2,3-Propanetriyl tris(12- | Elementis |
| | hydroxyoctadecanoate | |
| Vaseline | Petrolatum, meets specification | Sigma-Aldrich |
| | of Ph. Eur. | |
| Silicone fluid | Dimethicone NF | Dow Corning |
| 20 cSt | | |
| ST silicone | Hexamethyldisiloxane | Dow Corning |
| 0.65 cSt | | |
| ST silicone | Octamethyltrisiloxane | Dow Corning |
| 1 cSt | | |
| Cyclomethicone | Decamethylcyclopentasiloxane | Dow Corning |
| 5 NF | | |

Daptomycin is sourced commercially or wet-milled to give particles within the desired size range.

### General Methods

### Formulation Preparation

### Method A. Preparation of Anhydrous Topical Suspension Formulations for Physical Inspection

A one-pot homogenisation preparation method was developed for prototype suspension formulations. Any volatile components/excipients (e.g. hexamethyldisiloxane (0.65 cSt), octamethyltrisiloxane or cyclomethicone) were incorporated into the formulation following homogenisation.

Formulations were prepared on either a 10 g or 15 g scale in 20 ml glass vial (Chromacol, UK) on a w/w basis (using either 2-place or 5-place laboratory balances). All components, excluding any volatile excipients, were transferred (solids first) to a single vial. Vial contents were homogenised for a total of 4 minutes (PowerGen 700 with a 7 mm probe on setting 4, ˜10,000 rpm, 2×2 minutes with vial cooling between sessions). Any volatile components were added (by weight) directly to the formulation vial. Vials were immediately tightly capped and shaken.

### Method B. Prototype Anhydrous Topical Suspension Formulations Containing Thixcin R for Physical Inspection (Example 5; Formulations S to U Described Herein)

To avoid unnecessary heating of daptomycin during the preparation of formulations containing Thixcin R the following approach below was used. Formulations were prepared on a 10 g scale in 20 ml glass vial (Chromacol, UK) on a w/w basis (using either 2-place or 5-place laboratory balances).

Daptomycin was wet milled to produce a fine dispersion by mixing under high shear for between 30 minutes and 1 hour to form a daptomycin concentrate.

Formulation components, excluding the dispersed daptomycin and associated Labrafac PG, were transferred to a vial and heated to 90-95° C. with stirring (magnetic follower). Earlier investigations indicated that elevated temperature appeared to provide improved results compared to Thixcin R preparation at ˜45-50° C. For formulation S described herein, the mixture was homogenised for a total of 4-5 minutes. For formulations T and U described herein homogenisation at elevated temperature was not used (to reduce foaming) and the mixtures were instead stirred for ≧30 minutes. The Thixcin R formulation mixtures were then allowed to cool under stirring to ˜50° C. At an appropriate time the wet milled daptomycin and associated Labrafac PG was warmed to ˜50° C. with stirring and 1.344 ml added via Microman positive displacement pipette to the Thixcin R mixture and the formulation mixed (magnetic follower) at that temperature for a short period until homogeneous. The selected volume was chosen to correspond to an addition of 1.4 g daptomycin/Labrafac PG but subsequent calculations indicated that this was equivalent to ˜1.29 g (and thus daptomycin loading was ˜1.87%). Formulation S was allowed to cool to room temperature under stirring. Formulation T was homogenized for a total of 4 minutes at ˜50° C. then allowed to cool under stirring. Formulation U was homogenized for a total of 2 minutes at ˜50° C., allowed to cool under stirring, then homogenized for a further 4 minutes at room temperature.

### Method C. Preparation of Anhydrous Topical Suspension Formulations for Stability Assessment (Example 1; Formulations A to D and F to H Described Herein)

Formulations were prepared as follows. All non-volatile components excluding daptomycin and a proportion of the IPM (present in the wet milled API) were transferred to a 20 ml glass vial. The vial containing wet milled API was vigorously stirred (magnetic follower) and appropriate quantities were transferred (via a positive displacement pipette and Pasteur pipette) to each formulation vial (via weight). Each suspension formulation (Formulation A-D, F-H) was then homogenised for four minutes with vial cooling. Any volatile components (Formulations F-H) were then added, and the suspensions briefly homogenised (10 seconds), capped and shaken.

### Example 1

### Preparation of Formulations A to D and F to H

The formulations outlined in Tables 2 and 3 below were each prepared on a 15 g scale using Method C outlined above. Fresh quantities of daptomycin were transferred from the bulk container and water content was measured (Aquamax KF Coulometric) in triplicate and the absolute purity of the daptomycin calculated (92.7% and 92.3% for Tables 2 and 3, respectively). The daptomycin content of each formulation was adjusted to a target 2% using the calculated absolute purity.

An example of the component weights (for Formulations F-H) is shown in Table 4.

TABLE 2
Non-aqueous suspension formulations for informal
stability assessment (15 g batches, Formulations
A-D suspensions, Formulation E ointment)

| | Formulation | Formulation | Formulation | Formulation |
|---|---|---|---|---|
| Component | A % (w/w) | B % (w/w) | C % (w/w) | D % (w/w) |
| Daptomycin* | 2 (2.16) | 2 (2.16) | 2 (2.16) | 2 (2.16) |
| Labrafac PG | 12 | 12 | — | — |
| IPM | 26 | 27 | 35 | 36 |
| Labrafac | 45.84 | 46.84 | 53.84 | 54.84 |
| lipophile | | | | |
| Capryol 90 | 5 | 5 | — | — |
| Ca stearate | 2 | — | 2 | — |
| Kollidon CL-M | 5 | 5 | 5 | 5 |
| Span 20 | 2 | 2 | 2 | 2 |
| Vaseline | — | — | — | — |

*API content corrected for absolute purity (chemical purity and measured water content)

TABLE 3
Non-aqueous suspension formulations for informal stability
assessment (15 g batches, Formulations F-H suspensions)

| | Formulation | Formulation | Formulation |
|---|---|---|---|
| Component | F % (w/w) | G % (w/w) | H % (w/w) |
| Daptomycin* | 2 (2.17) | 2 (2.17) | 2 (2.17) |
| IPM | 15 | 15 | 15 |
| Labrafac lipophile | 13.83 | 13.83 | 13.83 |
| Ca stearate | 2 | 2 | 2 |
| Kollidon CL-M | 5 | 5 | 5 |
| Span 20 | 2 | 2 | 2 |
| Silicone fluid 20 cSt | 15 | 15 | — |
| ST silicone 0.65 cSt** | 30 | 45 | 35 |
| Cyclomethicone 5 NF** | 15 | — | 25 |

*API content corrected for absolute purity (chemical purity and measured water content)
**Volatile components

TABLE 4
Construction procedure for prototype non-aq suspension formulations
F-H containing 2% daptomycin (15 g preparation scale)

| | Formulation | Formulation | Formulation |
|---|---|---|---|
| | F | G | H |
| Component/phase | Weight (g) | Weight (g) | Weight (g) |
| Phase A (to be | | | |
| added to B) | | | |
| Wet milled API in | 2.032 | 2.032 | 2.032 |
| IPM (16% w/w) | | | |
| IPM component | 1.707 | 1.707 | 1.707 |
| Daptomycin | 0.325* (0.300) | 0.325* (0.300) | 0.325* (0.300) |
| component | | | |
| Phase B | | | |
| Kollidon CL-M | 0.750 | 0.750 | 0.750 |
| Ca stearate | 0.300 | 0.300 | 0.300 |
| Span 20 | 0.300 | 0.300 | 0.300 |
| IPM | 0.543 | 0.543 | 0.543 |
| Labrafac lipophile | 2.075 | 2.075 | 2.075 |
| Silicone fluid 20 cSt | 2.250 | 2.250 | 0.000 |
| Phase C (added post | | | |
| homogenisation) | | | |
| ST silicone 0.65 cSt** | 4.500 | 6.750 | 5.250 |
| Cyclomethicone | 2.250 | 0.000 | 3.750 |
| 5 NF** | | | |
| Total (g) | 15.000 | 15.000 | 15.000 |

*API content corrected for absolute purity (chemical purity and measured water content)
** Volatile components

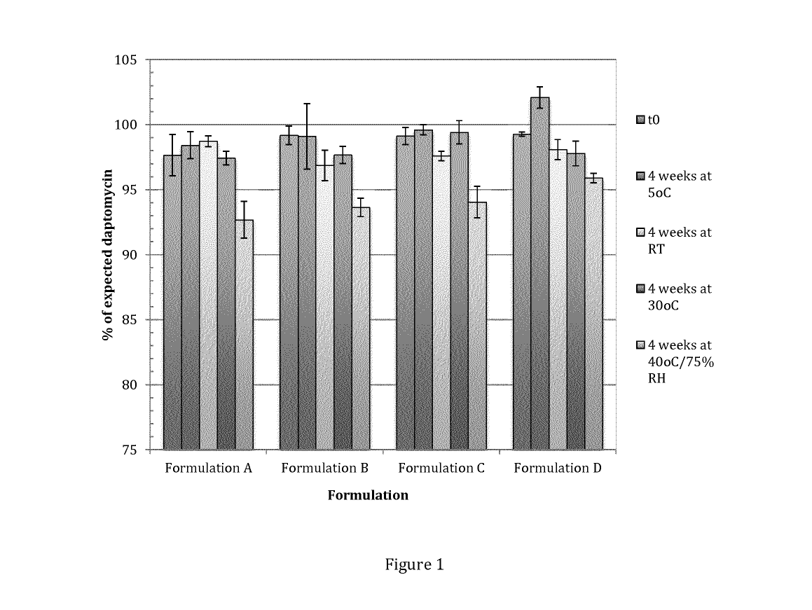

The percentage recovery of daptomycin from Formulations A-D over time is shown in FIG. 1.

### Example 2

### Preparation of Formulations I to L

Using the procedure described in Method A above, the following formulations I to L were prepared:

TABLE 5
Non-aqueous suspension formulations I to L

| | Formulation | Formulation | Formulation | Formulation |
|---|---|---|---|---|
| Component | I % (w/w) | J % (w/w) | K % (w/w) | L % (w/w) |
| Daptomycin | 2 | 2 | 2 | 2 |
| Labrafac PG | 12 | 12 | 12 | 12 |
| IPM | 34 | 34 | 28 | 32 |
| Captex 355 | 48 | 48 | 48 | 48 |
| Ca stearate | — | 1 | 2 | 2 |
| Capmul GMO | — | — | 5 | — |
| Kollidon CL-M | — | 1 | 1 | 2 |
| Span 20 | — | — | — | 2 |
| Span 80 | 3 | 2 | 2 | — |
| Tween 80 | 1 | — | — | — |

### Example 3

### Preparation of Formulations M to O

Using the procedure described in Method A above, the following formulations M to O were prepared:

TABLE 6
Non-aqueous suspension formulations M to O

| | Formulation | Formulation | Formulation |
|---|---|---|---|
| Component | M % (w/w) | N % (w/w) | O % (w/w) |
| Daptomycin | 2 | 2 | 2 |
| Labrafac PG | 12 | 12 | 12 |
| IPM | 27 | 29 | 29 |
| Labrafac lipophile | 48 | 48 | 48 |
| Capryol 90 | 5 | 5 | 5 |
| Ca stearate | 2 | 2 | — |
| Kollidon CL-M | 2 | — | 2 |
| Span 20 | 2 | 2 | 2 |

### Example 4

### Preparation of Formulations P to R

Using the procedure described in Method A above, the following formulations P to R were prepared:

TABLE 7
Non-aqueous suspension formulations P to R

| | Formulation | Formulation | Formulation |
|---|---|---|---|
| Component | P % (w/w) | Q % (w/w) | R % (w/w) |
| Daptomycin | 2 | 2 | 2 |
| Labrafac PG | 12 | — | — |
| IPM | 27 | 35 | 35 |
| Labrafac lipophile | 47 | 57 | 54 |
| Capryol 90 | 5 | — | — |
| Ca stearate | — | 2 | 2 |
| Kollidon CL-M | 5 | 2 | 5 |
| Span 20 | 2 | 2 | 2 |

### Example 5

### Preparation of Formulations S to U

Using the procedure described in Method B above, the following formulations S to U were prepared:

TABLE 8
Non-aqueous suspension formulations S to U

| | Formulation | Formulation | Formulation |
|---|---|---|---|
| Component | S % (w/w) | T % (w/w) | U % (w/w) |
| Daptomycin* | 2 | 2 | 2 |
| Labrafac PG | 12 | 12 | 12 |
| IPM | 29.4 | 29.7 | 25.8 |
| Labrafac lipophile | 47 | 47 | 46 |
| Capryol 90 | 5 | 5 | 5 |
| Ca stearate | 2 | 2 | 2 |
| Thixcin R | 0.6 | 0.3 | 0.2 |
| Span 20 | 2 | 2 | 2 |
| Kollidon CL-M | — | — | 5 |

*daptomycin milled at 14.3% in Labrafac PG (060213)

### Example 6

### Preparation of Formulations V and W

Using the procedure described in Method A above, the following formulations V and W were prepared:

TABLE 9
Non-aqueous suspension formulations V and W

| | Formulation | Formulation |
|---|---|---|
| Component | V % (w/w) | W % (w/w) |
| Daptomycin | 2 | 2 |
| IPM | 24 | 19 |
| Labrafac lipophile | 15 | — |
| Ca stearate | 2 | 2 |
| Kollidon CL-M | 5 | 5 |
| Span 20 | 2 | 2 |
| Silicone fluid 20 cSt | 20 | 20 |
| ST silicone 0.65 cSt | 20 | 30 |
| ST silicone 1 cSt | 10 | 20 |

### Example 7

### Preparation of Formulation X

Using the procedure described in Method A above, the following formulation X was prepared:

TABLE 10
Non-aqueous suspension formulation X

| | Formulation |
|---|---|
| Component | X % (w/w) |
| Daptomycin | 2 |
| IPM | 15 |
| Labrafac lipophile | 14 |
| Ca stearate | 2 |
| Kollidon CL-M | 5 |
| Span 20 | 2 |
| Silicone fluid 20 cSt | 15 |
| ST silicone 0.65 cSt | 45 |

### Example 8

### Preparation of Formulation Y

Using the procedure described in Method A above, the following formulation Y was prepared:

TABLE 11
Non-aqueous suspension formulation Y

| | Formulation |
|---|---|
| Component | Y % (w/w) |
| Daptomycin | 2 |
| IPM | 15 |
| Labrafac lipophile | 14 |
| Ca stearate | 2 |
| Kollidon CL-M | 5 |
| Span 20 | 2 |
| Silicone fluid 20 cSt | 10 |
| ST silicone 0.65 cSt | 40 |
| Cyclomethicone 5 NF | 10 |

### Example 9

### Preparation and Assessment of Formulations Z and AA

Using the procedure described in Method A above, the following formulations Z and AA were prepared:

TABLE 12
Non-aqueous suspension formulations Z and AA

| | Formulation | Formulation |
|---|---|---|
| Component | Z % (w/w) | AA % (w/w) |
| Daptomycin | 2 | 2 |
| IPM | 15 | 15 |
| Labrafac lipophile | 14 | 14 |
| Ca stearate | 2 | 2 |
| Kollidon CL-M | 5 | 5 |
| Span 20 | 2 | 2 |
| Silicone fluid 20 cSt | 15 | 0 |
| ST silicone 0.65 cSt | 30 | 30 |
| Cyclomethicone 5 NF | 15 | 30 |

### Example 10

### Preparation of Formulations BB and CC

Using the procedure described in Method A above, the following formulations BB and CC were prepared:

TABLE 13
Non-aqueous suspension formulations BB and CC

| | Formulation | Formulation |
|---|---|---|
| Component | BB % (w/w) | CC % (w/w) |
| Daptomycin | 2 | 2 |
| IPM | 15 | 15 |
| Labrafac lipophile | 14 | 14 |
| Ca stearate | 2 | 2 |
| Kollidon CL-M | 5 | 5 |
| Span 20 | 2 | 2 |
| Silicone fluid 20 cSt | 5 | 0 |
| ST silicone 0.65 cSt | 45 | 35 |
| ST silicone 1 cSt | 0 | 0 |
| Cyclomethicone 5 NF | 10 | 25 |

### Example 11

### Assessment of Sedimentation Volumes

Formulations were prepared for physical assessment on 10 g or 15 g scales as described above in reference to Examples 1 to 10 and were visually assessed over various time-periods for sedimentation and re-suspendability of solids.

Formulations were stored in 20 ml glass vials and the height of the solids/liquids monitored. Sedimentation volume, F, was estimated using the liquid (V₀) and solid heights (V_{H}) measured using vernier calipers, with the vial base height subtracted from each measurement prior to calculation. For example, Formulation F described herein at 5 days, F=V_{H}/V₀=13.9 mm/31.8 mm=˜44%, and upon shaking (hand-shaken 3 times) solids were easily re-suspended. Further assessment at 10 days and 24 days produced F values of ˜40% and ˜48% respectively, and solids continued to be easily re-suspended.

Approximate sedimentation volumes for the Formulations A to CC at various time points are shown in Table 14 below.

TABLE 14
approximate sedimentation values

| | Days since | Approx. sedimentation |
|---|---|---|
| Formulation | preparation | volume |
| A | 28 | 57% |
| | 96 | 68% |
| B | 28 | 51% |
| | 96 | 58% |
| C | 28 | 67% |
| | 96 | 61% |
| D | 28 | 62% |
| | 96 | 63% |
| F | 7 | 58% |
| G | 7 | 55% |
| H | 7 | 55% |
| I | 1 | 33% |
| J | 1 | 22% |
| | 154 | 18% |
| K | 1 | 30% |
| | 154 | 27% |
| L | 1 | 52% |
| | 2 | 50% |
| | 154 | 40% |
| M | 1 | 46% |
| | 4 | 45% |
| | 7 | 41% |
| | 12 | 42% |
| | 27 | 43% |
| | 153 | 38% |
| N | 1 | 44% |
| | 4 | 38% |
| | 27 | 29% |
| | 153 | 23% |
| O | 1 | 51% |
| | 4 | 43% |
| | 27 | 39% |
| | 153 | 40% |
| P | 3 | 65% |
| | 6 | 59% |
| | 11 | 60% |
| | 26 | 57% |
| | 155 | 53% |
| Q | 3 | 51% |
| | 6 | 39% |
| | 11 | 47% |
| | 26 | 41% |
| | 155 | 35% |
| R | 2 | 71% |
| | 7 | 65% |
| | 22 | 66% |
| | 151 | 46% |
| S | 124 | ~83% |
| T | 1 | very small amount of sedimentation |
| | 9 | very small amount of sedimentation |
| | 113 | not separated |
| U | 3 | 69% |
| | 105 | 70% |
| V | 1 | 56% |
| | 4 | 54% |
| | 11 | 52% |
| | 25 | 54% |
| W | 1 | 47% |
| | 4 | 40% |
| W | 11 | 38% |
| | 25 | 44% |
| X | 3 | 44% |
| | 10 | 40% |
| | 24 | 48% |
| Y | 31 | 44% |
| Z | 32 | 43% |
| AA | 1 | 48% |
| | 6 | 48% |
| | 17 | 46% |
| BB | 3 | 47% |
| | 14 | 42% |
| CC | 3 | 48% |
| | 14 | 41% |

All formulations could be re-suspended.

### Example 12

### Spreading Assessments

Selected formulations (Formulations A-D and F-G) were assessed for spreadability on glass surfaces and human skin.

Glass petri dishes (90 mm diameter) were located above a ruler and 100 μl of formulation applied in the centre of the petri dish. The spreading (extent and speed) for the assessed formulations was recorded and assessed. By comparison, water (100 μl) applied in an identical manner produced a discrete drop of ˜11 mm diameter which remained static between 10 seconds and 25 minutes. For formulations A-D described herein, the formulation spread rapidly and both the solid and liquid components reached a diameter of ˜18-21 mm over a period of ≦15 seconds and the diameter remained in that range for a further 25 minutes. For formulations F-G described herein, the formulation spread rapidly and solid components reached a diameter of ˜38-43 mm and the liquid components a diameter of ˜43-52 mm over a period of ˜34-37 seconds, then appeared static.

Application to human skin was assessed by transferring a (10 μl) drop to human skin (horizontal) and observing the self-spreading of the formulation over 2 minutes. By comparison, water (10 μl) applied in an identical manner produced a discrete drop of ˜3 mm diameter (˜0.07 cm²) which remained static for the 2 minute observation period. Formulations A-D spread rapidly without further intervention from the applicator and by 10 seconds each formulation had covered ≧0.3 cm² and this increased further to ≧0.5 cm² over the 2 minutes, at which point the rate of spreading had slowed greatly. Formulations F-G spread rapidly without further intervention from the applicator and by 10 seconds each formulation had covered ≧1 cm² and this increased further over the 2 minutes, ranging from ˜2 to 5 cm², at which point the rate of spreading had slowed greatly due to the considerable loss of volatile components.

What is claimed is:

1. A pharmaceutical composition suitable for topical application to the body, said composition comprising an antibacterial agent that is dissolved and/or suspended within an oily vehicle;
wherein the pharmaceutical composition is spontaneously spreadable upon application to a surface of the body.

2. The pharmaceutical composition according to claim 1, wherein the antimicrobial agent is uniformly dispersed or dispersible within the oily vehicle.

3. The pharmaceutical composition according to claim 1, wherein the antimicrobial agent is present as discrete particles.

4. The pharmaceutical composition according to claim 3, wherein the antimicrobial agent particles have a D90 of 75 μm or less.

5. The pharmaceutical composition according to claim 1, wherein the antimicrobial agent is present in an amount of 0.01 wt. % to 10 wt. % of the total composition.

6. The pharmaceutical composition according to claim 1, wherein the antimicrobial agent is selected from the group consisting of daptomycin, vancomycin, mupirocin, polymixin B, clindamycin, retapamulin, neomycin, bacitracin and fusidic acid, or a pharmaceutically acceptable salt thereof.

7. The pharmaceutical composition according to claim 6, wherein the antimicrobial agent is agent is selected from daptomycin, vancomycin and/or mupirocin or a pharmaceutically acceptable salt thereof.

8. The pharmaceutical composition according to claim 1, wherein the oily vehicle comprises one or more oil components selected from the group consisting of mono-, di- and/or triglycerides comprising glycerol and (6-18C) fatty acids; propylene glycol esters of (6-18C) fatty acids; (6-18C) fatty acid esters; (6-18C) fatty alcohols; silicone fluids/oils; and lanolin or a lanolin derivative.

9. The pharmaceutical composition according to claim 1, wherein the oily vehicle constitutes between 70 wt. % and 99.5 wt. % of the composition.

10. The pharmaceutical composition according to claim 1, wherein the oily vehicle comprises an oily emollient and said emollient constitutes up to 70% wt. % of the composition.

11. The pharmaceutical composition according to claim 10, wherein the oily vehicle comprises one or more oil components selected from the group consisting of mono-, di- and/or tri-glycerides comprising glycerol and (6-18C) fatty acids; propylene glycol esters of (6-18C) fatty acids; (6-18C) fatty acid esters (e.g. isopropyl myristate); and silicone fluids/oils.

12. The pharmaceutical composition according to claim 10, wherein the oily vehicle comprises 5-45 wt. % (6-18C) fatty acid esters; 5-60 wt. % mono-, di- and/or tri-glycerides comprising glycerol and (6-18C) fatty acids; 0-10 wt. % propylene glycol esters of (6-18C) fatty acids; 0-70 wt. % silicone fluids/oils; and 0-5 wt. % of a lanolin or a lanolin derivative.

13. The pharmaceutical composition according to claim 10, wherein the composition oily vehicle further comprises a suspending agent.

14. The pharmaceutical composition according to claim 13, wherein the suspending agent is selected from the group consisting of a cross-linked polymer; a clay; magnesium/aluminium silicate; a silica; and hydrogenated oil.

15. The pharmaceutical composition according to claim 14, wherein the suspending agent is crospovidone.

16. The pharmaceutical composition according to claim 13, wherein the suspending agent is present in an amount of 0.2-15 wt. % of the composition.

17. The pharmaceutical composition according to claim 10, wherein the oily vehicle further comprises one or more oily surfactants.

18. The pharmaceutical composition according to claim 17, wherein the oily surfactant is present in an amount of up to 7.5 wt. % of the total composition.

19. The pharmaceutical composition according to claim 17, wherein the oily surfactant has a HLB (hydrophilic-lipophilic balance) value of less than 7.

20. The pharmaceutical composition according to claim 17, wherein the surfactant is selected from the group consisting of polyoxyethylated fatty acid (10-18C) esters of sorbitan (e.g. Span 20, Span 80); (8-18C) fatty acid esters, and polyoxypropylene/polyoxyethylene block co-polymers (Poloxamers®).

21. The pharmaceutical composition according claim 1, wherein the pharmaceutical composition comprises a suspending agent and an oily surfactant.

22. The pharmaceutical composition according to claim 1, wherein the composition further comprises a divalent metal salt stabilizer, optionally selected from calcium fatty acid salts, magnesium fatty acid salts, calcium sulfate, magnesium sulfate, calcium trisilicate, magnesium trisilicate, calcium carbonate and magnesium carbonate.

23. The pharmaceutical composition according to claim 22, wherein the amount of stabilizer present is 0-5 wt. % of the total composition.

24. The pharmaceutical composition according to claim 1, wherein the moisture content of the compositions of the present invention is less than 1 wt. %.

25. The pharmaceutical composition according to claim 1, wherein the composition comprises:
0.05-5 wt. % of an antibacterial agent;
75-99.5 wt. % of an oily vehicle; and
optionally 0.5-10 wt. % of a suspending agent.

26. The pharmaceutical composition according to claim 1, wherein the composition comprises:
0.05-5 wt. % of daptomycin or a pharmaceutically acceptable salt thereof;
5-45 wt. % (6-18C) fatty acid esters;
5-60 wt. % mono-, di- and/or triglycerides comprising glycerol and (6-18C) fatty acids;
0-10 wt. % propylene glycol esters of (6-18C) fatty acids;
0-70 wt. % silicone oils;
0-5 wt. % lanolin/lanolin derivative;
up to 7.5 wt. % of a surfactant selected from the group consisting of polyoxyethylated fatty acid (10-18C) esters of sorbitan; (8-18C) fatty acid esters, and polyoxypropylene/polyoxyethylene block co-polymers;
0.5-10 wt. % of a suspending agent selected from the group consisting of cross-linked polymers suitably in a micronized form; a clay; magnesium/aluminium silicates; silica; and hydrogenated oil; and
0-5 wt. % of a divalent metal salt.

27. The pharmaceutical composition according to claim 1, wherein the composition comprises:
0.2-4 wt. % of daptomycin or a pharmaceutically acceptable salt thereof;
10-40 wt. % isopropyl myristate;
10-60 wt. % caprylic/capric triglyceride/medium chain triglyceride;
0-30 wt. % dimethicone;
0-40 wt. % hexamethyldisiloxane;
0-30 wt. % cyclomethicone;
1-5 wt. % of a surfactant selected from the group consisting of polyoxyethylated fatty acid (10-16C) esters of sorbitan; (8-16C) fatty acid esters, and polyoxypropylene/polyoxyethylene block co-polymers;
1-8 wt. % of crospovidone, sodium croscarmellose and trihydroxystearin; and
1-3 wt. % of a divalent calcium and/or magnesium salt.

28. The pharmaceutical composition according to claim 1, for use in the treatment of a topical infection infections.

29. The pharmaceutical composition according to claim 28, wherein said topical infection is selected from the group consisting of impetigo, MRSA and a complicated skin or skin structure infection (cSSSI).

30. The pharmaceutical composition according to claim 1, for use in the eradication of a topical MRSA prior to surgery.

31. A method of treating a topical infection, said method comprising administering to a human or animal subject in need of such treatment a therapeutically effective amount of a pharmaceutical composition according to claim 1.

32. A method of treating MRSA or a complicated skin and/or skin structure infection (cSSSI), said method comprising administering to a human or animal subject in need of such treatment a therapeutically effective amount of a pharmaceutical composition according to claim 1.

33. A method of eradicating topical MRSA prior to surgery, said method comprising administering to a human or animal subject in need of such treatment a therapeutically effective amount of a pharmaceutical composition according to claim 1.

34. A method of preparing a pharmaceutical composition according to claim 1, said method comprising suspending an antibacterial agent in an oily vehicle.

35. A device comprising a pharmaceutical composition according to claim 1, wherein the device is configured to dispense a volume of the composition for topical application.
